(19) United States
(12) Patent Application Publication
(10) Pub. No.: US 2013/0096428 A1
(43) Pub. Date: Apr. 18, 2013

(54) ACCESS SYSTEM FOR FEMORAL VASCULATURE CATHETERIZATION AND RELATED METHOD

(71) Applicants: George T. Gillies, Charlottesville, VA (US); Srijoy Mahapatra, Charlottesville, VA (US); D. Scott Lim, Charlottesville, VA (US); Michael Ragosta, Charlottesville, VA (US)

(72) Inventors: George T. Gillies, Charlottesville, VA (US); Srijoy Mahapatra, Charlottesville, VA (US); D. Scott Lim, Charlottesville, VA (US); Michael Ragosta, Charlottesville, VA (US)

(73) Assignee: University of Virginia Patent Foundation, Charlottesville, VA (US)

(21) Appl. No.: 13/579,745

(22) PCT Filed: Dec. 21, 2010
(86) PCT No.: PCT/US10/61413

§ 371 Date: Dec. 24, 2012

Related U.S. Application Data
(60) Provisional application No. 61/305,152, filed on Feb. 17, 2010.

(51) Int. Cl. A61M 25/01

(52) U.S. Cl. CPC A61M 25/01
USPC 600/434

(57) ABSTRACT

An access system and method for obtaining access to the interior vascular structures or other regions or collections of fluid or fluid-filled cavities inside the body. The system and method provides for injection of contrast agents (to confirm ideal position or condition), the passage of guide wires, and the eventual catheterization of the heart and other parts of the body via the pathway established through the puncture of a femoral artery. The system and method provides the ability to inject a contrast material and pass a guide wire through the same introducer device simultaneously (without necessarily moving it or removing any parts), with the device designed to prevent the backflow of the contrast material through the guide wire port during the contrast injection process The ideal location of access in the vein or artery can be seen by injecting contrast from a needle inside the structure and then using fluoroscopy.

### RELATED APPLICATIONS

The present application claims priority from U.S. Provisional Application Ser. No. 61/305,152, filed Feb. 17, 2010, entitled “Access Device for Femoral Vasculature Catheterization and Method of Use;” the disclosure of which is hereby incorporated by reference herein in its entirety.

### FIELD OF INVENTION

The present invention relates generally to the field of medical devices to be used for cardiological procedures. More specifically, the invention is in the subfield of vascular access devices and other tools to be used for cardiac and endovascular catheterizations, including, for example, ablation and in electrophysiological procedures.

### BACKGROUND OF THE INVENTION

The femoral artery is the predominant site of vascular access for the performance of cardiac catheterizations, percutaneous coronary interventions and many other vascular related procedures. The operator's goal is to safely and reliably puncture a certain part of the common femoral artery. Several palpable anatomic landmarks are used to guide the operator, however, these landmarks are notoriously imprecise and the exact puncture site is not known until after a sheath is inserted and an angiogram performed. Failure to properly puncture the common femoral artery may result in several important and potentially life-threatening complications. In the event of a puncture above the inguinal ligament (i.e. in the external iliac artery), a retroperitoneal bleed may result. If the puncture is too low and in one of the branches of the common femoral artery, then a hematoma, femoral artery pseudoaneurysm or arteriovenous fistulae may arise. All together, these events complicate up to 5% of cardiac procedures and cause significant morbidity, patient discomfort, increased mortality, greater hospital costs and prolonged length of stay.

There are many examples of access devices or systems that are used to carry out

percutaneous puncture of a femoral artery or vein for the purpose of catheterizing the heart or some other part of the body. However, despite a myriad of prior art, access to the femoral vein and artery are complicated in 5% of all cases by hematoma or other vascular complication, especially during cardiac interventions when large doses of anticoagulants (blood thinners) are used. This is often because the prior needle systems routinely access the femoral vein or artery either too high (leading to retroperitoneal bleeding) or too low (leading to femoral vein or artery bleeds, pseudoaneurysms, or arteriovenous fistulae). The ideal location of the access in the vein or artery can be seen by injecting contrast from a needle inside the structure and then using fluoroscopy. However, a major limitation of the prior art is that it does not allow for an easy method to inject a contrast material into the artery to confirm the needle position in the common femoral artery or vein while simultaneously maintaining a method for introducing a guide wire. Under the prior art, the operator would have to access the vein, then inject contrast agent, remove the contrast injection means (being hopeful that the needle doesn't move) and then insert a wire through the needle. Introducing this wire would be difficult since the needle opening is so small.

A further limitation of the prior art is that such introducer means are generally not small enough to easily reposition the needle during the process of insertion of it into and passage through the body tissues. The smaller the needle the smaller the potential damage, as Pouiselle's Law suggests the damage and potential for bleeding is dependent on the inverse fourth power of the needle's radius. However, in the prior art the smaller needle requires an even smaller guide wire to be loaded.

Still another limitation of the prior art is that such introducer means can cause unnecessary and dangerous bleeding during usage or may led to catheter placement either into one of the smaller branches of the common femoral artery or in a deeper, more proximal artery (e.g. the external iliac artery), potentially resulting in an increased rate of serious vascular complications or bleeding.

An additional limitation of the prior art is that after the chamber has been loaded with contrast agent, the chamber cannot be cleared of air in the chamber. The injection of contrast fluid with air bubbles can cause serious medical complications.

An additional limitation of the prior art is that the needle means incorporated into the introducer system kits generally do not have markings on them to indicate depth of insertion, nor do the needles have markings that are usually visible on fluoroscopic means for monitoring the needle insertion process.

The prior art is silent on the teaching of a means and method for overcoming these limitations and thus achieving an improved technique for percutaneous puncture of the common femoral artery for the purpose of passing a catheter through the vasculature to a target location in the heart or some other part of the body.

### BRIEF SUMMARY OF THE INVENTION

An aspect of an embodiment of the present invention relates to, but not limited thereto, the class of medical devices used to obtain access to the interior vascular structures or other regions or collections of fluid or fluid-filled cavities inside the body. More specifically, an aspect of an embodiment of the present invention relates to, but not limited thereto, that class of percutaneous puncture means that allow for the injection of contrast agents (to confirm ideal position), the passage of guide wires, and the eventual catheterization of the heart and other parts of the body via the pathway established through the puncture of a femoral artery. Therefore, an aspect of an embodiment of the present invention relates to, but not limited thereto, percutaneous access devices that target entry into a vascular structure (such as the femoral artery and vein) or another region or part of the body, and that have the ancillary means to enable safe and efficacious accomplishment thereof.

An aspect of an embodiment of present invention provides a means and method for, but not limited thereto, the injection of a contrast material and the passage of a guide wire through the same introducer device simultaneously (without necessarily moving it or removing any parts), with the device designed so as to prevent the backflow of the contrast material through the guide wire port during the contrast injection process. The needle or related components of the access system may be fabricated in such a way that the user can measure the insertion depth by visual inspection of markings on it during the insertion process. For needles made of materials not other wise readily visible by fluoroscopy, the markings would be radio-opaque and thus the needle location and depth could be determined by reference to fluoroscopic imaging.

An aspect of an embodiment of the present invention provides the capability of injecting a contrast agent while simultaneously navigating a guide wire into an artery. The access system is not limited to accessing an artery. The access system may be used to access the vicinity of a vasculature, the vasculature, or the non-vasculature structure of a subject. The non-vasculature structure includes, but is not limited to, a gall bladder, kidney, cyst, pus collection, or some other normal or diseased organ, body part, or other element of the body. Additionally, the vasculature includes arteries and veins within the endovascular system. This includes the femoral artery and a radial artery. The access system can be designed so that the components of the system are transparent so that the user of the system can see a backsplash of blood regurgitating through the access needle following successful insertion of the access needle into a vasculature. The access system can also be designed so that the components of the access system are MRI compatible so that the access system can be used with MR-guided interventional procedures. The assembly is leak-tight to the contrast agent, blood, saline irrigants, and other fluids.

Another aspect of an embodiment of the present invention is, but not limited thereto, the ability to reduce the probability that a user will access the femoral vein or artery either too high (leading to retroperitoneal bleeding) or too low (leading to femoral vein or artery bleeds, pseudoaneurysms, or arteriovenous fistulae). The ideal location of the access in the vein or artery can be seen by injecting contrast from a needle inside the structure and then using fluoroscopy. An aspect of an embodiment of the present invention allows a user to minimize the probability of unnecessary and dangerous bleeding during usage and minimize the probability of catheter placement either into one of the smaller branches of the common femoral artery or in a deeper, more proximal artery (e.g. the external iliac artery), potentially resulting in an increased rate of serious vascular complications or bleeding.

Another aspect of an embodiment of the present invention is that a user can reposition the needle during the process of inserting the needle into and through the body tissues. An additional advantage of an embodiment of the present invention is that after the chamber has been loaded with contrast agent, the chamber can be cleared of air in the chamber to minimize the likelihood of injecting contrast fluid with air bubbles. An aspect of an embodiment of the present invention can also be equipped with a needle that includes markings to indicate depth to assist in locating and guiding the needle

Another aspect of an embodiment of the present invention is the capability of injecting a contrast agent while simultaneously navigating a guide wire into an artery. The contrast agent can be loaded into the syringe body and the plunger can be advanced to push the contrast agent through a chamber and out of the access needle. The chamber can be, for example but not limited thereto, a y-tube connector or a syringe body. The chamber may be configured to allow the guide wire to pass through the chamber and the access needle while simultaneously injecting the contrast agent from the chamber into the subject to guide the access needle beneath the skin, in a non-vasculature structure, in the vicinity of a vasculature and/or in the vasculature. Fluoroscopy can be used to determine the location of the access needle and guide wire. The access needle can be equipped with markings so that the user can determine how far the access needle has been inserted into the subject.

Another aspect of an embodiment of the present invention is the ability to, but not limited thereto, seal off one of the arms of the y-tube when the chamber is implemented with, for example, a y-tube (or elbow or arm). A valve can be used to accomplish this task. The valve can be designed to allow a user to manually open and close the value and allow the valve to be closed when the guide wire is passed through the valve. When the valve is closed, contrast agent will not leak out of the valve when the contrast agent is being injected into the subject. Additionally, the valve effectively holds the guide wire in place when it is closed. A septum can also be used in combination with an introducer conduit to accomplish substantially the same task as the valve.

An another aspect of an embodiment of the present invention is, but not limited thereto, that the assembly of components, when properly configured, provides for a means and method of injecting a contrast agent through the access needle and also passing a guide wire through the same needle simultaneously in such a way that the contrast agent does not back-flow through the port into which the guide wire enters the assembly and does not require removing of any parts once access is obtained. The assembly can be leak-tight to the contrast agent, blood, saline irrigants, and other fluids.

Another aspect of an embodiment of the present invention is the ability to, but not limited thereto, use of a sheath. The sheath may be designed to prevent damage to the vasculature or non-vasculature structure when the access needle and/or other devices are inserted into, moved within, or removed from the vasculature or non-vasculature structure.

Another aspect of an embodiment of the present invention is, but not limited thereto, the ability to at least partially fill the chamber with contrast agent and then clear the chamber of air. This prevents air bubbles from potentially being injected into the subject when the contrast agent is injected.

Another aspect of an embodiment of the invention present is the use of, for example finger loops or ergonomic grips or handles. Finger loops allow for easy ergonomic use of the access system during procedures. The finger loop fittings make it possible to either inject or withdraw the plunger using only one hand, because the fingers can work against either the upper or lower inner surfaces of the finger loops.

Another aspect of an embodiment of the present invention is the use of a slip fitting or other releasable fittings for attaching the access needle to the access system. This allows for rapid and easy attachment and detachment of the access needle from the system.

Another aspect of an embodiment of the present invention is a third arm on the y-tube when a y-tube is used to implement the chamber. This permits another material, device or another liquid to be injected or another device to be passed into the body in conjunction with the delivery of the contrast agent and passage of the guide wire.

Another aspect of an embodiment of the present invention provides, but not limited thereto, a cardiological approach for vascular access devices and other tools to be used for cardiac and endovascular catheterizations.

Another aspect of an embodiment of the present invention provides, but not limited thereto, the ability to safely and reliably puncture a certain part of the common femoral artery.

An aspect of an embodiment of the present invention provides, but not limited thereto, an access system that provides the capability to inject a contrast agent while simultaneously navigating a guide wire into a vicinity of a vasculature, a non-vasculature structure, and/or the vasculature of a subject through a tip of an access needle. The access system may comprise: the access needle; the guide wire; and a chamber. The chamber may be configured to allow the guide wire to pass through the chamber and the access needle while simultaneously injecting the contrast agent from the chamber into the subject to guide the access needle beneath the skin, in the vicinity of a vasculature, the non-vasculature structure, and/or in the vasculature.

An aspect of an embodiment of the present invention provides, but not limited thereto, a method for using an access system that provides the capability to inject a contrast agent while simultaneously navigating a guide wire into a vasculature, a non-vasculature structure, and/or a vicinity of a vasculature of a subject through a tip of an access needle. The method may comprise: advancing the access needle of the system through the skin of a subject; determining if the access needle is in the correct location; and a) if the access needle is in the correct location then further advancing the access needle of the system until the access needle has accessed the vasculature, the non-vasculature structure, and/or the vicinity of the vasculature of the subject; or b) if the access needle is not in the correct location withdrawing or repositioning the access needle and then determining if the access needle in the correct location.

An aspect of an embodiment of the present invention provides, but not limited thereto, a method for using an access system that provides the capability to inject contrast agent while simultaneously navigating a guide wire into a vicinity of a vasculature, a non-vasculature structure, and/or the vasculature of a subject through a tip of an access needle. The access method may comprise: passing the guide wire through a chamber and the access needle while simultaneously injecting contrast agent from the chamber into the subject to guide the access needle that is inserted beneath the skin, in the vicinity of a vasculature, non-vasculature and/or in the vasculature.

These and other objects, along with advantages and features of various aspects of embodiments of the invention disclosed herein, will be made more apparent from the description, drawings and claims that follow.

### BRIEF DESCRIPTION OF THE DRAWINGS

The foregoing and other objects, features and advantages of the present invention, as well as the invention itself, will be more fully understood from the following description of the embodiments, when read together with the accompanying drawings.

The accompanying drawings, which are incorporated into and form a part of the instant specification, illustrate several aspects and embodiments of the present invention and, together with the description herein, serve to explain the principles of the invention. The drawings are provided only for the purpose of illustrating select embodiments of the invention and are not to be construed as limiting the invention.

FIG. 1 schematically illustrates a system view of the access system.

FIG. 2 schematically illustrates an embodiment of the access system.

FIG. 3 schematically illustrates an embodiment of the access system.

FIG. 4 schematically illustrates the use of finger loops which can be implemented with any embodiment of the access system.

FIG. 5 schematically illustrates an embodiment of the access system.

FIG. 6A schematically illustrates an embodiment of the access system prior to puncturing the skin.

FIG. 6B schematically illustrates the access system after the access needle has punctured the skin.

FIG. 6C schematically illustrates the access system after the sheath and access needle have punctured the skin.

FIG. 6D schematically illustrates the access system after the access needle is removed from the subject and the sheath is left in place after it punctures the skin.

FIG. 6E illustrates the access system after the access needle punctures a vasculature or non-vasculature structure.

FIG. 6F schematically illustrates the access system after the access needle and the sheath puncture a vasculature or non-vasculature structure.

FIG. 6G illustrates the access system after the access needle is removed from the subject and the sheath is left in place after it punctures a vasculature or non-vasculature structure.

FIG. 7 provides a flowchart representing a method of an embodiment for obtaining percutaneous puncture of a vasculature or non-vasculature structure.

FIG. 8A provides a flowchart representing a method of an embodiment for obtaining percutaneous puncture of a vasculature or non-vasculature structure.

FIG. 8B provides a flowchart representing a method of an embodiment for determining if the access needle is in the correct location, as required by, for example, the method in FIG. 8A.

### DETAILED DESCRIPTION OF THE INVENTION

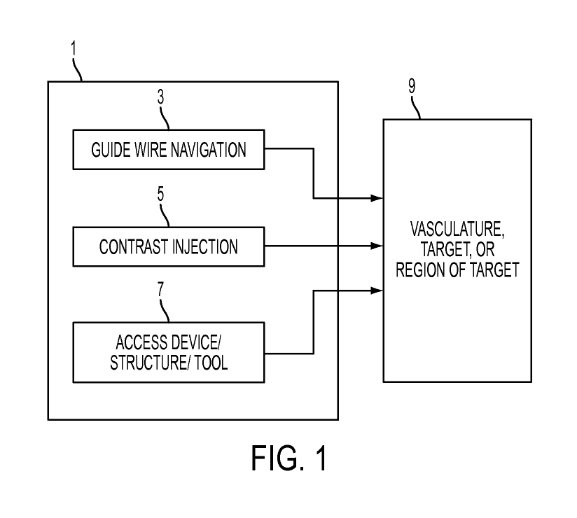

FIG. 1 is a schematic illustration of access system 1 that provides the capability of injecting 5 a contrast agent while simultaneously navigating a guide wire 3 and an access structure/device/tool 7 into a target 9 such as a blood vessel or region of the target of a subject. The target 9 may include a vasculature or non-vasculature structure and the region of the target includes the area in the vicinity of the vasculature or non-vasculature structure. The access system 1 is not limited to accessing an artery. The access system 1 may be used to access the vicinity of a vasculature, the vasculature, or the non-vasculature structure of a subject. The non-vasculature structure includes, but is not limited to, a gall bladder, kidney, cyst, pus collection, or some other normal or diseased organ, body part, or other element of the body. Additionally, the vasculature includes arteries and veins within the endovascular system. This includes, but not limited thereto, the femoral artery and a radial artery. The access system 1 can be designed so that the components of the system are transparent so that the user of the system can see a backsplash of blood regurgitating through the access needle following successful insertion of the access needle into a vasculature. The access system 1 can also be designed so that the components of the access system 1 are MRI compatible so that the access system 1 can be used with MR-guided interventional procedures. The components that can be MRI compatible and/or transparent include for example, but are not limited to, the access needle, the chamber, introducer conduit, plunger, and the guide wire. These components will be explained more with reference to the subsequent figures. MRI compatible indicates, for example, (1) that the device or components thereof being inserted into the MRI will not move under the influence of the MRI's magnetic field, (2) that the device or components thereof being inserted into the MRI will not create an “imaging artifact” that affects the diagnostic or therapeutic quality of the MRI image, and (3) that the patient won't be harmed by either magnetically-induced movement or heating of the device being inserted into the MRI.

Fluoroscopy can be used with the contrast agent to navigate the guide wire 3 and any components of the access system 1 beneath the skin, in a non-vasculature structure, in the vicinity of a vasculature, and/or in the vasculature. Additionally, an optical guide wire can be used.

It should be appreciated that connectors/fittings used in the access system 1 may be integral to the components themselves or the connectors/fittings may be separate components. It should also be appreciated that any of the components may be attached directly or integral with other components to avoid a particular fitting/connector.

It should be appreciated that as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g. rat, dog, pig, monkey), etc. It should be appreciated that the subject may be any applicable human patient, for example.

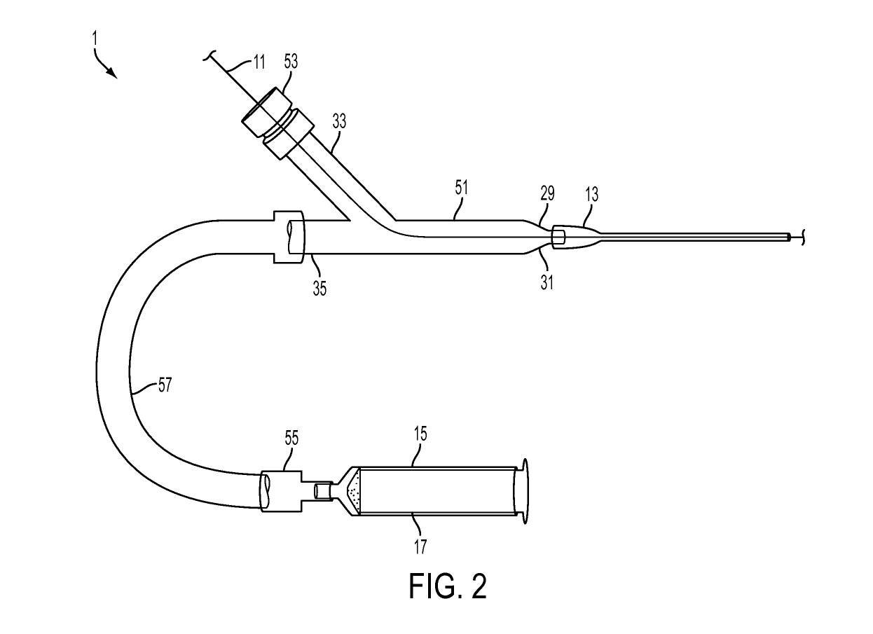

FIG. 2 schematically shows an embodiment of access system 1. The contrast agent (not shown) is loaded into the syringe body 15 and the plunger 17 can be advanced to push the contrast agent from the contrast agent reservoir of the syringe body 15 into the tube 57 whereby a connector 55 may be implemented. It should be appreciated that the tube and syringe body may connect directly or integral thus eliminating the connector. The contrast fluid is delivered to the chamber 51 through the chamber second proximal end 35 via the connection tube 57, thus making it possible to locate the access system 1 anywhere convenient within the surgical field, including areas where access with a standard syringe is typically more difficult or prohibited. As shown in FIG. 2, the chamber 51 is a y-tube. In another embodiment, the chamber 51 may also include a third proximal end (not shown) for applications where another material, device or another liquid is injected or another device must be passed into the body in conjunction with the delivery of the contrast agent and passage of the guide wire 11.

As shown in FIG. 2, the chamber 51 is configured to allow the guide wire 11 to pass through the chamber 51 and the access needle 13 while simultaneously injecting the contrast agent from the chamber 51 into the subject to guide the access needle beneath the skin, in a non-vasculature structure, in the vicinity of a vasculature and/or in the vasculature. In an approach, a valve 53 may be attached to the chamber first proximal end 33. The valve 53 can be Tuohy-Borst connector or other types of available connectors. The valve 53 disposed on the chamber first proximal end 33 can be closed to seal off the chamber first proximal end 33. The guide wire 11 can be inserted through the valve 53. The valve 53 can be closed when the guide wire 11 or another tool is being passed through the valve 53. Additionally, the valve 53 can be closed by the user manually. The valve 53 can substantially prevent contrast agent from leaking out of the valve 53 when the contrast agent is being injected into the subject. Additionally, the valve 53 may effectively holds the guide wire 11 in place when it is closed. In an exemplary operation, the guide wire 11 may be placed next to the access system 1 and is ready to be inserted into the valve 53 and passed through the distal tip of the access needle 13. The chamber first proximal end 33 is configured to receive the guide wire 11 to allow for the guide wire to be passed through the chamber distal end 31 and into the access needle 13. The guide wire 11 can then be passed though the tip of the access needle 13. The chamber distal end 31 can be designed to have conical shape nose. The conical shape nose on the chamber distal end 31 guides the guide wire from the chamber 51 into the access needle 13 and can prevent the guide wire 11 from turning back on itself and getting jammed in the chamber 51. In an embodiment, the taper angle of the conical shape nose of the chamber distal end 31 would be in the range from about 45° to 50°, although other angles can also be used. FIG. 2 serves merely as an example of one embodiment of the access system 1, and the specific depictions, contours and dimensions therein do not serve as limitations; these components may be implemented in a number of different ways.

In an approach, the access needle 13 may be in communication with the chamber distal end 31 via a Luer slip fitting or other type of fitting or connector for rapid and easy attachment and detachment of the access needle 13 from the system 1. Any other type of fitting known in the art can be used to attach the access needle 13 to the chamber distal end 31. The access needle 13 can be chosen from any of a number of commercially available such needles, for instance, but not limited thereto, the model NDL-100-22 from Galt Medical. The syringe body 15 and plunger 17 can be chosen from any of a number of commercially available syringes, for instance, but not limited thereto, the model 309642 from Becton Dickinson. The tube 57 can be chosen from any of a number of commercially available tubes, for instance, but not limited thereto, the model 536040 from Medex. The chamber can be chosen from any of a number of commercially available Y-connectors, for instance, but not limited thereto, the model 84044 from Qosina.

The access needle 13 may include marks located along the length of the needle. For example, the marks may be located at approximately 1 cm intervals or any desired intervals. The marks can be put in place by a number of means and methods, for instance, the placement of 1-cm long segments of silicone tubing along the needle's shaft, with the subsequent marking (e.g., via a biocompatible paint) of the 1-cm intervals. In some embodiments, the shaft of the access needle 13 may be made from a material that is un-opaque to x-rays and which would thus have little visibility upon fluoroscopic imaging. In such a case, the fiducial marks along the shaft of the access needle 13 could be of a radio-opaque material thus being clearly visible upon fluoroscopic or CT imaging. The access needle 13 can also include more than 1 internal lumen to facilitate the simultaneous or sequence delivery of more than one diagnostic or therapeutic agent, fluid, or device.

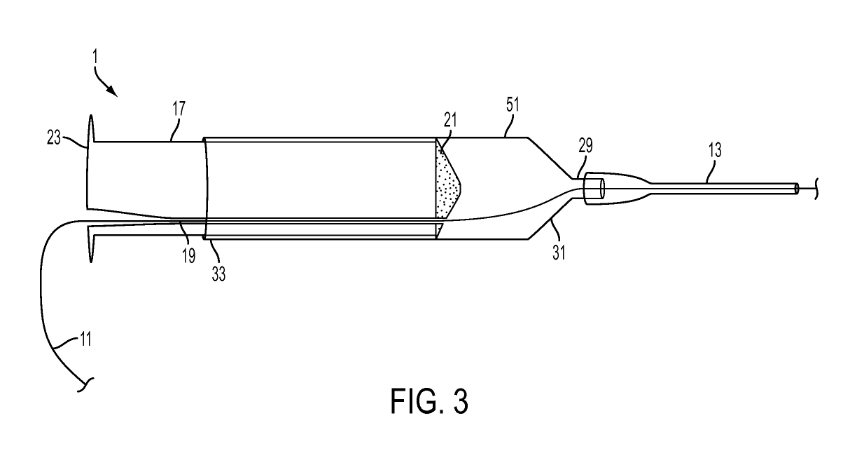

FIG. 3 schematically shows an embodiment of the access system apparatus 1. The chamber 51 is configured to allow the guide wire 11 to pass through the chamber 51 and the access needle 13 while simultaneously injecting the contrast agent from the chamber 51 into the subject to guide the access needle beneath the skin, in a non-vasculature structure, in the vicinity of a vasculature and/or in the vasculature. The chamber 51 includes a distal end 31 and a proximal end 33. The access needle 13 is in communication with the chamber distal end 31. A Luer slip fitting or other connector (not shown) may be used for rapid and easy attachment and detachment of the access needle from the chamber 51 or system 1. Any other type of fitting known in the art can be used to attach the access needle 13 to the chamber distal end 31. The chamber proximal end 33 may be configured to receive the guide wire 11 to allow for the guide wire to be passed through the chamber distal end 31 and into the access needle 13. The guide wire 11 can then be passed though the tip of the access needle 13.

The chamber proximal end 33 may be configured to receive a plunger 17. The plunger 17 includes an introducer conduit 19 that extends from the plunger proximal end 23 to the plunger distal end 21. The introducer conduit includes a proximal end configured to receive the guide wire 11 and a distal end configured to pass the guide wire 11 into the chamber 51 and through the access needle 13. The chamber distal end 31 can be designed to have conical shape nose. The conical shape nose on the chamber distal end 31 guides the guide wire from the chamber 51 into the access needle 13 and can prevent the guide wire 11 from turning back on itself and getting jammed in the chamber 15.

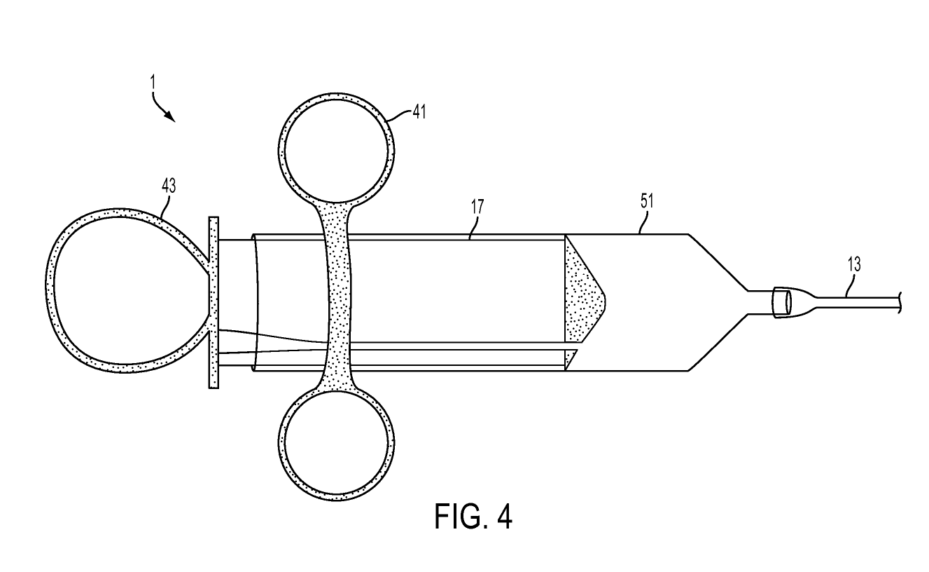

FIG. 4 schematically illustrates the access system 1 from FIG. 3, however the access system 1 includes syringe body finger loops 41 and plunger finger loops 43. Finger loop fittings 41 and 43 can be permanently attached to the system or alternatively slid onto or connect to the proximal ends of the chamber 51 and the plunger 17 to make for easy ergonomic use of the syringe during access procedures. The finger loop fittings 41 and 43 make it possible to either inject or withdraw the plunger using only one hand, because the fingers can work against either the upper or lower inner surfaces of the finger loops.

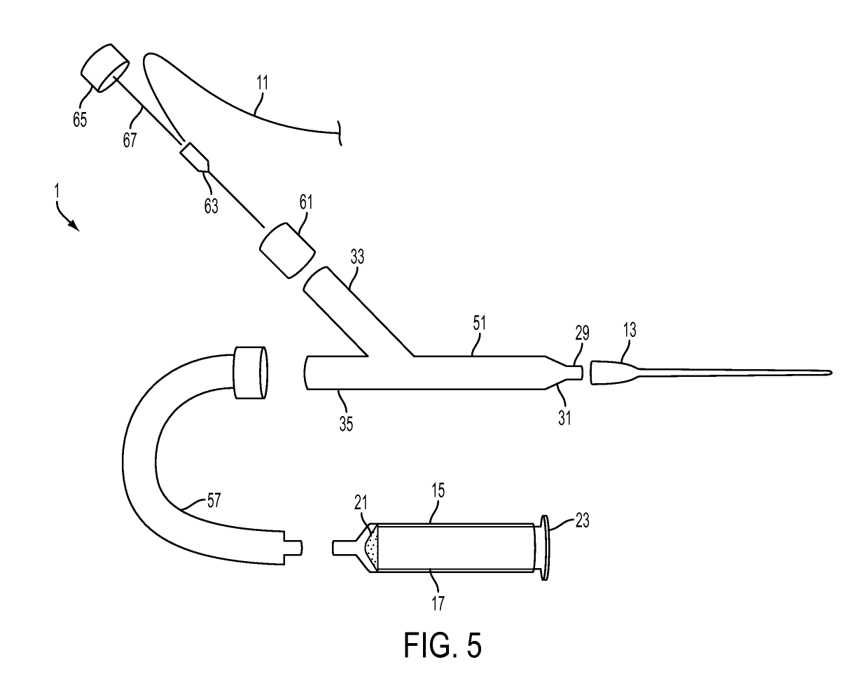

FIG. 5 schematically shows an embodiment of system apparatus 1. The system apparatus 1 can be used to deliver contrast material into the chamber 51. The contrast agent (not shown) is loaded into the syringe body 15 and the plunger 17, with plunger distal end 21 and plunger proximal end 23, can be advanced to push the contrast agent from the contrast agency reservoir of the syringe body 15 into the tube 57. The contrast fluid is delivered to the chamber 51 through the chamber second proximal end 35 via inter-connection tube 57, thus making it possible to locate the system apparatus 1 anywhere convenient within the surgical field including areas where access with a standard syringe is typically more difficult or prohibited. As shown in FIG. 5, the chamber 51 may be a y-tube. In another embodiment, although not shown, the chamber 51 may still yet include a third proximal end for applications where another material or another liquid is injected or another device must be passed into the body in conjunction with the delivery of the contrast agent and passage of the guide wire 11.

The chamber 51 is configured to allow the guide wire 11 to pass through the chamber 51 and the access needle 13 while simultaneously injecting the contrast agent from the chamber 51 into the subject to guide the access needle beneath the skin, in a non-vasculature structure, in the vicinity of a vasculature and/or in the vasculature. A septum 61, a Luer-locked septum in one embodiment, may be attached to the chamber first proximal end 33. It should be appreciated that a variety of fittings may be implemented. In an approach, fixed onto the distal entry port of the septum 61, closest to the chamber 51, is a seal (not shown) that serves as the seat against which the lower outside bowl of an introducer conduit 63 is seated following insertion of the tip of the introducer conduit 63 through the seal internal to the septum 61. The introducer conduit 63 allows a guide wire 11 to be passed through the septum 61, the seal, and into the chamber 51. As shown in FIG. 5, the introducer conduit 63 may be a needle stub. The seal can be made of a barrier material made from one of a number of different substances, including latex, silicone, or other such materials appropriate for use in injection site fittings. The seal can be glued in place, threaded into place or, alternatively, the septum 61 can be manufactured with the equivalent of a seal element integral to the upper portion of the septum 65. The seal substantially prevents contrast agent from leaking out of the septum 61 when the contrast agent is being injected into the subject.

A cap 65 can be used to seal the chamber first proximal end 33. The cap 65 can include a stylet 67, which can be advanced through the introducer conduit 63. In an exemplary operation, a guide wire 11 may be placed next to the assembly of the system and is ready to be inserted into the introducer conduit 63 and passed through the distal tip of the access needle 13. The chamber first proximal end 33 is configured to review the guide wire 11 to allow for the guide wire 11 to be passed through the chamber distal end 31 and into the access needle 13. The guide wire 11 can then be passed though the tip of the access needle 13. The chamber distal end 31 can be designed to have conical shape nose. The conical shape nose on the chamber distal end 31 guides the guide wire 11 from the chamber 51 into the access needle 13 and can prevent the guide wire 11 from turning back on itself and getting jammed in the chamber 15. In an approach, the access needle 13 may be in communication with the chamber distal end 31 via a Luer slip fitting or other connection type for rapid and easy attachment and detachment of the access needle 13 from the system. Any other type of fitting known in the art can be used to attach the access needle 13 to the chamber distal end 31.

The septum 61 may be chosen from among any of a number of commercially available septums, for instance, but not limited thereto, model 84039 from Qosina. The seal (not shown) of the septum 61 may be a o-ring that may be chosen from any of a number of commercially available o-ring means, for instance, but not limited thereto, model KNW-876 from GC Electronics. The introducer conduit 63 might be chosen from any of a number of commercially available needle stubs, for instance, but not limited thereto, model 6710A28 from McMaster-Carr. The stylet 67 can be fabricated from any of a number of commercially available small gauge tubes or wires, for instance, but not limited thereto, model HTX-26R-12 from Small Parts, Inc. The cap 65 can be chosen from any of a number of commercially available stylet caps, for instance model 71301 from Qosina.

Still referring to FIG. 5, the chamber 51 could include a third proximal end (not shown) for applications where another material or another liquid is injected or another device must be passed into the body in conjunction with the delivery of the contrast agent and passage of the guide wire 11.

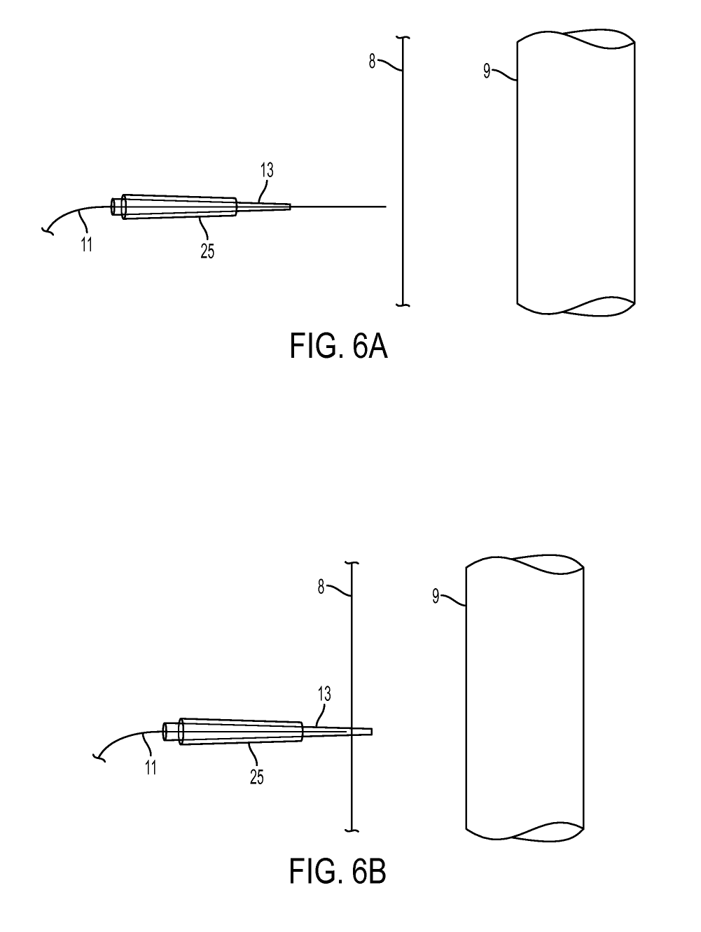

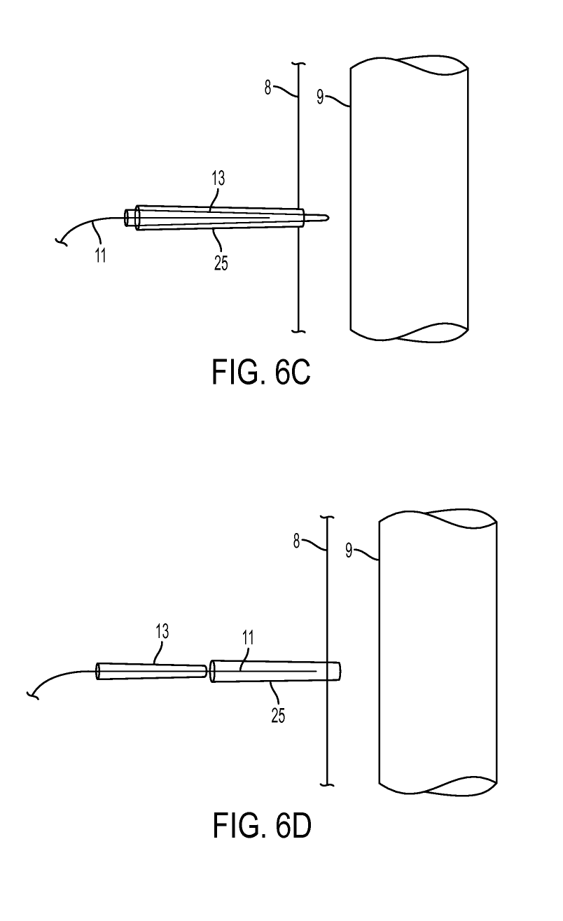

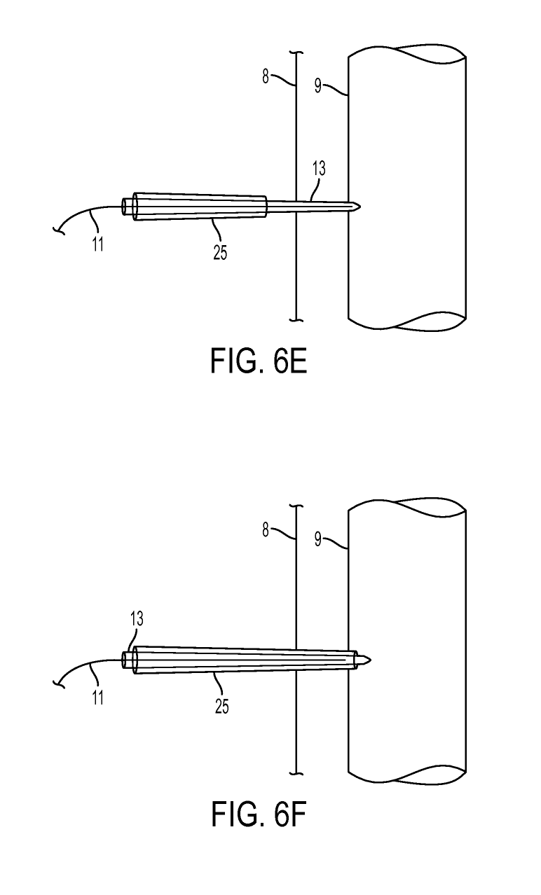

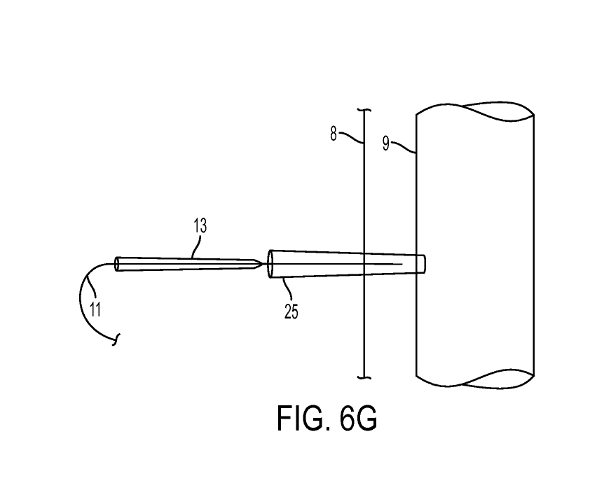

FIGS. 6A, 6B, 6C, 6D, 6E, 6F, and 6G schematically illustrate the use of a sheath 25 with the access needle 13 as it pertains to an aspect of an embodiment of the present invention system and method. While FIGS. 6A, 6B, 6C, 6D, 6E, 6F, and 6G illustrate a vasculature 9, it should be appreciated that the vasculature 9 can be a non-vasculature or alternatively the access needle 13 could just be in the region of either vasculature or non-vasculature, not actually puncturing it. FIG. 6A schematically illustrates the vasculature 9 with skin 8 before the access needle 13 punctures the skin 8. The access needle 13 is ready to puncture the skin 8 and the sheath 25 is already placed over or passed along the access needle 13. The guide wire 11 is shown as extending through the tip of the access needle 13, however the guide wire 11 does not need to be extended through the tip of the access needle 13 at this time. It should be appreciated that the system and method can be practiced without the sheath or, alternatively, a different device, material or tool substituted for the sheath as desired or required.

Unless clearly specified to the contrary, there is no requirement for any particular described or illustrated activity or element, any particular sequence or such activities, any particular size, speed, material, duration, contour, dimension or frequency, or any particularly interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Moreover, any activity or steps can be done in various orders, as well as sequentially, simultaneously or some combination thereof. It should be appreciated that aspects of the present invention may have a variety of sizes, contours, shapes, compositions and materials as desired or required.

It should be appreciated that various sizes, dimensions, contours, rigidity, shapes, flexibility and materials of any of the embodiments discussed throughout may be varied and utilized as desired or required.

FIG. 6B schematically illustrates the puncture of the skin 8 by the access needle 13. The access needle 13 has punctured the skin 8, but the sheath 25 has not punctured the skin 8 and the guide wire 11 has not been extended through the tip of the access needle 13. The guide wire 11 could be extended through the tip of the access needle 13 and contrast fluid (not shown) could be injected below the skin 8 to assist in guiding the access needle 13 and guide wire 11 using fluoroscopy. The guide wire 11 and the access needle would be visible to a user if contrast agent is injected below the skin and fluoroscopy is used to guide the access system. The user can extend the guide wire 11 through the tip of the access needle 13 so that the user can see where the guide wire 11 and the access needle 13 are located relative to a vasculature. The user can then adjust the location if necessary to ensure that the proper location of the vasculature or non-vasculature structure is accessed when the access needle 13 is advanced. The guide wire 11 allows the user, when using contrast agent and fluoroscopy, to slide the guide wire 11 through the tip of the access needle 13 so that the user doesn't risk accidentally puncturing a vasculature or non-vasculature in the wrong location. The user can determine the location of the guide wire 11, ensure that the guide wire 11 is located near the target area of the vasculature or non-vasculature structure, and then slide the access needle 13 over the guide wire 11 until the vasculature or non-vasculature structure 9 is accessed by the access needle 13.

FIG. 6C schematically illustrates the sheath 25 puncturing the skin 8. The access needle 13 has already punctured the skin 8 and now the sheath 25 has been slid or advanced forward so that the sheath punctures the skin 8. The guide wire 11 could be extended through the tip of the access needle 13 and contrast fluid could be injected below the skin 8 to assist in guiding the access needle 13 and guide wire 11 using fluoroscopy.

FIG. 6D schematically illustrates whereby the access needle 13 is removed from the skin 8 after the sheath 25 has punctured the skin. The sheath 25 is left in place, accessing below the skin 8, after the access needle 13 is removed.

FIG. 6E schematically illustrates whereby the access needle 13 punctures the vasculature 9 or non-vasculature structure. The sheath 25 has not been inserted into the skin 8 yet. The sheath 25 could be advanced into the skin 8 prior to the access needle 13 puncturing the vasculature 9 or non-vasculature structure.

FIG. 6F schematically illustrates whereby the sheath 25 is advanced along the access needle 13. The sheath 25 punctures the vasculature or non-vasculature structure 9.

FIG. 6G schematically illustrates when the access needle 13 is removed from the subject. The guide wire 11 is left in place below the skin 8 in the illustration. The guide wire 11 could be removed with, before, or after the access needle 13 is removed from the subject. After the access needle 13 is removed, the sheath is still accessing the vasculature or non-vasculature structure 9. The sheath 25 is designed to prevent damage to the vasculature or non-vasculature structure 9 when the access needle 13 and/or other devices are inserted into, moved within, or removed from the vasculature or non-vasculature structure 9. The other devices that can be inserted into the subject through the sheath 25 include, but are not limited to, various materials such as powders and slurries of diagnostic and therapeutic agents, other liquids such as antibiotics, anticoagulants, and thrombolytic agents, other guide wires or lumens, and other devices such as thrombectomy devices, closure devices, dilators, and sheaths.

It should be appreciated that any of the components or modules referred to with regards to any of the present invention embodiments discussed herein, may be a variety of materials and/or composites as necessary or required. Still further, it should be appreciated that any of the components or modules (or combination thereof) may provide shape, size and volume contoured by adjusting its geometry and flexibility/rigidity according to the target location or anatomy (or region, including structure and morphology of any location) being treated.

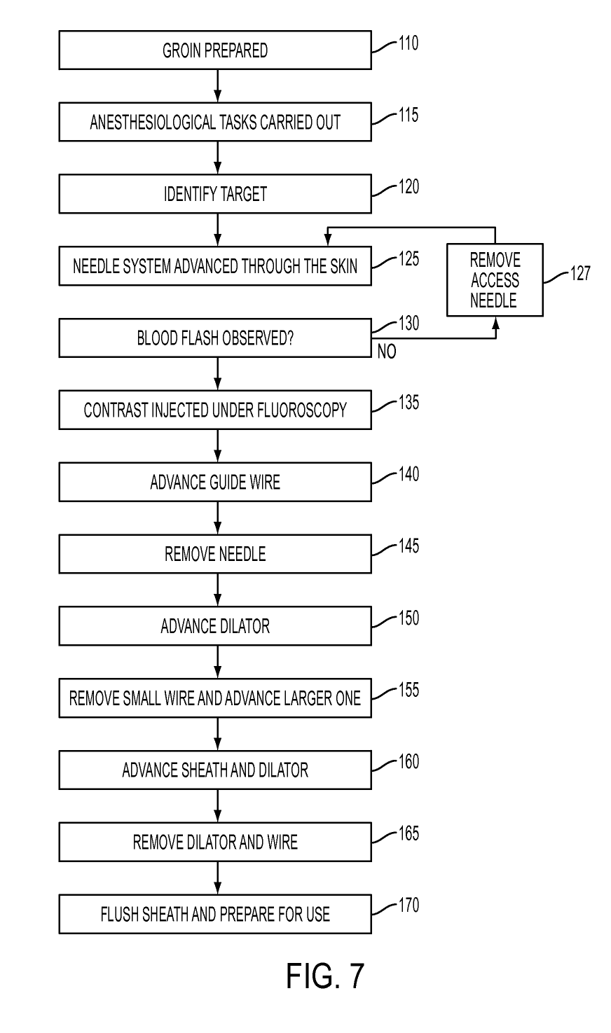

FIG. 7 provides a flowchart representing an embodiment of a method for obtaining a percutaneous puncture of a vasculature or non-vasculature structure. First, a groin area (not shown) is prepared 110 in the usual way known to a person having ordinary skill in the art. Anesthesiological tasks 115 are then carried out. Next a target vasculature or non-vasculature structure is identified as the target 120. The access needle of an access system is advanced through the skin 125 using fluoroscopic guidance and bony landmarks to position the needle at the appropriate location near the target. The access system will be preloaded with a contrast agent and a guide wire. The contrast agent is injected 135 after a blood flash is observed 130. If no blood flash is observed, the access system is removed 127 and the process starts over. The location of the access needle and guide wire can be determined using fluoroscopy. If the position of the access needle is not ideal, the access system can be withdrawn, the system can be flushed, and the user can begin the process from the beginning starting with advancing the access needle 125. If the positions of the guide wire and access needle are ideal, the guide wire can be advanced 140 further or the access needle can be advanced until the vasculature or non-vasculature is punctured. The access needle can then be removed 145. A dilator can be placed or advanced 150 over the guide wire, the guide wire can then be removed, a larger wire can be placed or advanced 155 via the dilator, a sheath and dilator can be advanced 160 over the larger wire, the dilator and wire can be removed 165, the sheath can be flushed 170, and now the medical procedure can be continued.

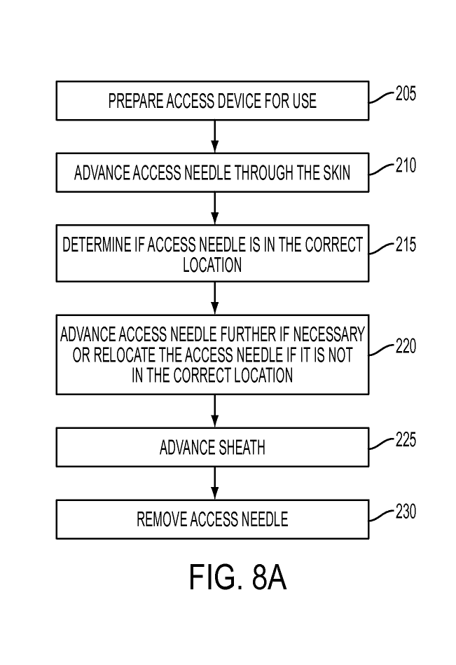

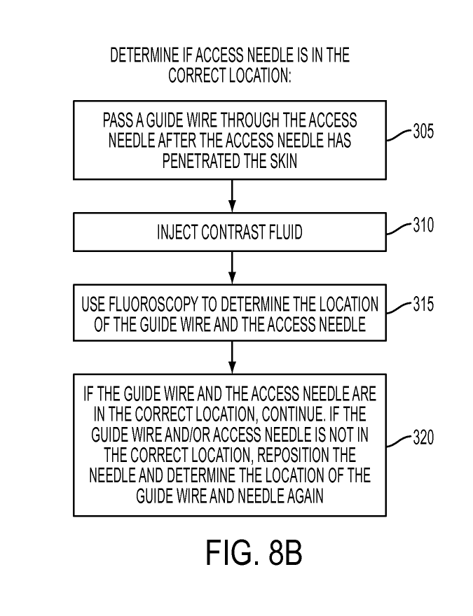

FIG. 8A provides a flowchart representing a method of an embodiment for obtaining a percutaneous puncture of a vasculature or non-vasculature structure. The access device is prepared for use 205. The access needle is advanced through the skin 210. The location of the access needle is determined 215. If the access needle is not in the correct location, the access needle is removed and the process begins again 212. If the access needle is in the correct location, the access needle is advanced 220 further until the access needle punctures the target vasculature or non-vasculature structure. The sheath is then advanced 225 until the sheath punctures the target vasculature or non-vasculature structure. The access needle can then be removed 230 and the vasculature or non-vasculature structure can be targeted as necessary for the specific procedure being performed. The method for using the access system provides, for example, but not limited thereto, the capability to inject a contrast agent while simultaneously navigating a guide wire into a vasculature, a non-vasculature structure, and/or a vicinity of a vasculature of a subject through a tip of an access needle.

FIG. 8B provides a flowchart representing a method of an embodiment for determining if the access needle is in the correct location, as required by the method in FIG. 8A. A guide wire is passed through the access needle after the access needle has penetrated the skin 305. The guide wire can be advanced through the chamber of the access system and the access needle while simultaneously injecting the contrast agent 310 from the chamber into the subject to guide the access needle beneath the skin, in the vicinity of a vasculature and/or in the vasculature

Contrast fluid is then injected 310 and fluoroscopy is used to determine the location of the guide wire and the access needle 315. If the guide wire and the access needle are in the correct location, the method of using the invention to obtain percutaneous puncture of a vasculature or non-vasculature structure can continue. If the guide wire and/or access needle is not in the correct location, the needle and guide wire are repositioned and the location is determined again 320. The guide wire allows the user to determine approximately where the access needle would puncture the target vasculature or non-vasculature structure. The access needle can then be advanced over the guide wire so that the access needle punctures the target vasculature or non-vasculature structure approximately where the guide wire was located when the location of the guide wire and the access needle was determined.

The various embodiments of the present invention provide, but not limited thereto, an easy, easy and safe method to inject a contrast material into the artery to confirm the needle position in the common femoral artery or vein while simultaneously maintaining a method for introducing a guide wire. The various embodiments of the present invention provide, but not limited thereto, a needle system that reduces the probability that a user will not access the femoral vein or artery either too high (leading to retroperitoneal bleeding) or too low (leading to femoral vein or artery bleeds, pseudoaneurysms, or arteriovenous fistulae). The ideal location of the access in the vein or artery can be seen by injecting contrast from a needle inside the structure and then using fluoroscopy. The various embodiments of the present invention allow, but not limited thereto, a user to minimize the probability of unnecessary and dangerous bleeding during usage and minimize the probability of catheter placement either into one of the smaller branches of the common femoral artery or in a deeper, more proximal artery (e.g. the external iliac artery), potentially resulting in an increased rate of serious vascular complications or bleeding.

The various embodiments of the present invention also allows, but not limited thereto, a user to reposition the needle during the process of insertion of it into and passage through the body tissues. An additional advantage of the various embodiments of the present invention is that after the chamber has been loaded with contrast agent, the chamber can be cleared of air in the chamber to minimize the likelihood of injecting contrast fluid with air bubbles. The various embodiments of the present invention can also be, but not limited thereto, equipped with a needle that includes markings to indicate depth to assist in locating and guiding the needle

### EXAMPLES

Practice of an aspect of an embodiment (or embodiments) of the invention will be still more fully understood from the following examples, which are presented herein for illustration only and should not be construed as limiting the invention in any way.

Example 1 includes an access system that provides the capability to inject a contrast agent while simultaneously navigating a guide wire into a vicinity of a vasculature, a non-vasculature structure, and/or the vasculature of a subject through a tip of an access needle, the access system comprising:

the access needle;

the guide wire; and

a chamber, wherein the chamber is configured to allow the guide wire to pass through the chamber and the access needle while simultaneously injecting the contrast agent from the chamber into the subject to guide the access needle beneath the skin, in the vicinity of a vasculature, the non-vasculature structure, and/or in the vasculature.

Example 2 may optionally include at least in part the system of example 1, wherein fluoroscopy is used with the contrast agent to navigate the guide wire and the access needle beneath the skin, in the vicinity of a vasculature, the non-vasculature structure, and/or in the vasculature.

Example 3 may optionally include at least in part the system of example 1, wherein the access needle comprises markings at intervals along its shaft for the purpose of indicating depth of insertion into the subject.

Example 4 may optionally include at least in part the system of example 1, wherein the vasculature comprises arteries and/or veins within the endovascular system.

Example 5 may optionally include at least in part the system of example 1, wherein the vasculature further comprises a femoral artery.

Example 6 may optionally include at least in part the system of example 1, wherein the vasculature further comprises a radial artery.

Example 7 may optionally include at least in part the system of example 1, wherein the non-vasculature structure comprises a gall bladder, kidney, cyst, pus collection, or some other normal or diseased organ, body part, or other element of the body.

Example 8 may optionally include at least in part the system of example 1, wherein the access needle has more than one internal lumen to facilitate the simultaneous or sequential delivery of more than one diagnostic or therapeutic agent, fluid, or device.

Example 9 may optionally include at least in part the system of example 1, wherein the access system further comprises:

a sheath, wherein the sheath is configured for sliding over the access needle.

Example 10 may optionally include at least in part the system of example 9, wherein the sheath is configured for insertion into the vasculature after the access needle punctures the vasculature, to prevent damage to the vasculature when the access needle and/or other devices are inserted into, moved within, or removed from the vasculature.

Example 11 may optionally include at least in part the system of example 1, wherein the chamber comprises a distal end and a first proximal end and a second proximal end;

the access needle is in communication with the chamber distal end;

the chamber first proximal end is configured for receiving the guide wire to allow for the passing of the guide wire through the chamber distal end and into the access needle.

Example 12 may optionally include at least in part the system of example 11, further comprising a contrast agent reservoir, wherein the contrast agent reservoir is in communication with the chamber.

Example 13 may optionally include at least in part the system of example 12, wherein the chamber second proximal end is in communication with the contrast agent reservoir.

Example 14 may optionally include at least in part the system of example 13, wherein the contrast agent reservoir comprises:

a plunger, wherein the plunger comprises a distal end and a proximal end; and

a syringe, wherein the syringe comprises a distal end and a proximal end, whereby the proximal end has an opening configured for receiving the plunger.

Example 15 may optionally include at least in part the system of example 12, wherein the communication between the chamber and the contrast reservoir comprises a tube.

Example 16 may optionally include at least in part the system of example 11, wherein the chamber distal end comprises a conical shape nose.

Example 17 may optionally include at least in part the system of example 16, wherein the conical shape nose comprises a Luer slip fitting on the conical shape nose, the Luer slip fitting capable of receiving the access needle for rapid and easy attachment and detachment of the access needle from the system.

Example 18 may optionally include at least in part the system of example 11, wherein at least one of the following components of the system are optically transparent so that a user can see a backsplash of blood regurgitating through the access needle following successful insertion of the access needle into the vasculature:

the access needle;

the chamber; or

the guide wire.

Example 19 may optionally include at least in part the system of example 11, wherein the chamber is a y-tube, the chamber first proximal end and the chamber second proximal end correspond to a first proximal arm and a second proximal arm of the y-tube respectively and the chamber distal end corresponds to a distal arm of the y-tube.

Example 20 may optionally include at least in part the system of example 19, wherein an angle between the first proximal arms and the second proximal arms is less than about 90 degrees.

Example 21 may optionally include at least in part the system of example 20, wherein the angle between the first proximal arms and the second proximal arms is approximately 30 degrees.

Example 22 may optionally include at least in part the system of example 11, wherein at least one of the following components of the system are MRI compatible so that the system can be used with MR-guided interventional procedures:

the access needle;

the chamber; or

the guide wire.

In Example 23 may optionally include at least in part the system of example 11, wherein the chamber further comprises a third proximal end for applications where other material or other liquid is injected or other device must be passed into the body in conjunction with the delivery of the contrast agent and passage of the guide wire.

Example 24 may optionally include at least in part the system of example 23, wherein:

the other material further comprises at least one of powders of diagnostic or therapeutic agents or slurries of diagnostic or therapeutic agents;

the other liquids further comprises at least one of antibiotics, anticoagulants, or thrombolytic agents; and

the other device further comprises at least one of thrombectomy devices, closure devices, dilators, or sheaths.

Example 25 may optionally include at least in part the system of example 11, further comprising a seal disposed in the chamber first proximal end and an introducer conduit in communication with the seal, whereby the introducer conduit configured to allow passing of the guide wire through the seal and into the chamber.

Example 26 may optionally include at least in part the system of example 25, wherein the seal comprises a septum.

Example 27 may optionally include at least in part the system of example 25, wherein the introducer conduit comprises a needle stub.

Example 28 may optionally include at least in part the system of example 25, further comprising a cap for sealing off the introducer conduit, the seal, or the chamber first proximal end.

Example 29 may optionally include at least in part the system of example 25, wherein the seal comprises a barrier material made from one of a number of different substances, including latex, silicone, or other such materials appropriate for use in injection site fittings.

Example 30 may optionally include at least in part the system of example 11, further comprising a valve disposed on the chamber first proximal end.

Example 31 may optionally include at least in part the system of example 30, wherein the valve disposed in the chamber first proximal end can be closed to seal off the chamber first proximal end.

Example 32 may optionally include at least in part the system of example 31, wherein the valve can be closed by the user manually.

Example 33 may optionally include at least in part the system of example 31, wherein the valve comprises a Tuohy-Borst connector.

Example 34 may optionally include at least in part the system of example 30, wherein the valve disposed in the chamber first proximal end can be closed to seal off the chamber first proximal end when the guide wire or other tool is being passed through the valve.

Example 35 may optionally include at least in part the system of example 34, wherein the valve can be closed by the user manually.

Example 36 may optionally include at least in part the system of example 35, wherein the valve comprises a Tuohy-Borst connector.

Example 37 may optionally include at least in part the system of example 1, wherein the chamber comprises a distal end and a proximal end;

the access needle is in communication with the chamber distal end; and

the chamber proximal end is configured for receiving the guide wire to allow for the passing of the guide wire through the chamber distal end and into the access needle.

Example 38 may optionally include at least in part the system of example 37, wherein the chamber distal end comprises a conical shape nose.

Example 39 may optionally include at least in part the system of example 38, wherein the conical shape nose comprises a Luer slip fitting on the conical shape nose, the Luer slip fitting capable of receiving the access needle for rapid and easy attachment and detachment of the access needle from the system.

Example 40 may optionally include at least in part the system of example 37, wherein the chamber proximal end is configured to receive a plunger; wherein the plunger comprises a distal end and a proximal end.

Example 41 may optionally include at least in part the system of example 40, wherein the plunger comprises a introducer conduit extending from the plunger proximal end to the plunger distal end, wherein the introducer conduit comprises a proximal end configured to receive the guide wire and a distal end configured to pass the guide wire into the chamber.

Example 42 may optionally include at least in part the system of example 41, wherein at least one of the following components of the system are transparent so that a user of the system can see a backsplash of blood regurgitating through the access needle following successful insertion of the access needle into the vasculature:

the access needle;

the chamber;

the introducer conduit;

the plunger; or

the guide wire.

Example 43 may optionally include at least in part the system of example 41, wherein at least one of the following components of the system are MRI compatible so that the system can be used with MR-guided interventional procedures:

the access needle;

the chamber;

the introducer conduit;

the plunger; or

the guide wire.

Example 44 includes a method for using an access system that provides the capability to inject a contrast agent while simultaneously navigating a guide wire into a vasculature, a non-vasculature structure, and/or a vicinity of a vasculature of a subject through a tip of an access needle, the method comprising:

advancing the access needle of the system through the skin of a subject;

determining if the access needle is in the correct location; and:
if the access needle is in the correct location then further advancing the access needle of the system until the access needle has accessed the vasculature, the non-vasculature structure, and/or the vicinity of the vasculature of the subject; or
if the access needle is not in the correct location withdrawing or repositioning the access needle and then determining if the access needle in the correct location.

Example 45 may optionally include at least in part the method of example 44, wherein the determining if the access needle is in the correct location further comprises passing the guide wire through a chamber and the access needle while simultaneously injecting the contrast agent from the chamber into the subject to guide the access needle beneath the skin, in the vicinity of the vasculature, the non-vasculature structure, and/or in the vasculature.

Example 46 may optionally include at least in part the method of example 44, further comprising advancing a sheath over the access needle after advancing the access needle through the skin, wherein the sheath is accessing the vasculature, the non-vasculature structure, and/or the vicinity of the vasculature of the subject.

Example 47 may optionally include at least in part the method of example 46, further comprising advancing the sheath towards a vasculature after the access needle has punctured the vasculature or the non-vasculature structure, wherein the sheath is accessing the vasculature, the non-vasculature structure, and/or the vicinity of the vasculature of the subject.

Example 48 may optionally include at least in part the method of example 47, further comprising advancing the sheath over the access needle after the access needle has punctured the vasculature or the non-vasculature structure, wherein the sheath is advanced until the vasculature or the non-vasculature structure is entered by the sheath.

Example 49 may optionally include at least in part the method of example 44, further comprising advancing the guide wire through the access needle after the access needle punctures the skin.

Example 50 may optionally include at least in part the method of example 49, wherein advancing the guide wire through the access needle after the access needle punctures the skin further comprises passing the guide wire through a chamber and the access needle while simultaneously injecting contrast agent from the chamber into the subject to guide the access needle beneath the skin, in the vasculature, the non-vasculature structure, and/or the vicinity of the vasculature of the subject.

Example 51 may optionally include at least in part the method of example 49, wherein the determining if the access needle is in the correct location further comprising:

injecting the contrast agent into the vasculature, the non-vasculature structure, and/or the vicinity of a vasculature of a subject through the tip of the access needle;

determining the location of the access needle using fluoroscopy; and

verifying that the access needle is in the correct location.

Example 52 may optionally include at least in part the method of example 51, wherein the injecting the contrast agent into the vasculature, the non-vasculature structure, and/or the vicinity of a vasculature of a subject through the tip of the access needle further comprises injecting the contrast agent into the vasculature, the non-vasculature structure, and/or the vicinity of a vasculature of a subject through the tip of the access needle while a guide wire is passing through the chamber.

Example 53 may optionally include at least in part the method of example 52, wherein the verifying that the access needle is in the correct location further comprises verifying that the guide wire and the access needle are in the correct location.

Example 54 may optionally include at least in part the method of example 49, wherein the advancing the access needle of the system until the access needle has accessed the vasculature, the non-vasculature structure, and/or the vicinity of the vasculature of the subject further comprises advancing the access needle over the guide wire until the access needle has punctured a vasculature.

Example 55 may optionally include at least in part the method of example 54, further comprising:

advancing a sheath over the access needle after advancing the access needle through the skin, wherein the sheath is accessing the vasculature, the non-vasculature structure, and/or the vicinity of the vasculature of the subject; and

removing the access needle from the subject and skin after advancing the sheath over the needle, wherein the sheath is still accessing the vasculature, the non-vasculature structure, and/or the vicinity of the vasculature of the subject.

Example 56 may optionally include at least in part the method of example 55, wherein the sheath is accessing the vasculature.

Example 57 may optionally include at least in part the method of example 55, wherein the guide wire remains below the skin after the access needle is removed.

Example 58 may optionally include at least in part the method of example 44, wherein the access system is accessing a vasculature structure such as a femoral artery or radial artery.

Example 59 may optionally include at least in part the method of example 44, wherein the access system is accessing the non-vasculature structure such as a gall bladder, kidney, cyst, pus collection, or some other normal or diseased organ, body part, or other element of the body.

Example 60 includes a method for using an access system that provides the capability to inject contrast agent while simultaneously navigating a guide wire into a vicinity of a vasculature, a non-vasculature structure, and/or the vasculature of a subject through a tip of an access needle, the access method comprising:

passing the guide wire through a chamber and the access needle while simultaneously injecting contrast agent from the chamber into the subject to guide the access needle that is inserted beneath the skin, in the vicinity of a vasculature, non-vasculature and/or in the vasculature.

Example 61 may optionally include at least in part the method of example 60, wherein the method further comprises:

sliding a sheath over the access needle.

Example 62 may optionally include at least in part the method of example 61, further comprising:

puncturing the vasculature with the access needle; and

inserting the sheath into the vasculature after the access needle punctures the vasculature, to prevent damage to the vasculature when the access needle and/or other devices are inserted into, moved within, or removed from the vasculature.

Example 63 may include a method of manufacturing the access system according to example 1. The method may be implemented with any technique or process (or combinations thereof) available to a practitioner in the field of manufacturing.

Example 64 may optionally include at least in part the system of example 1, further comprising a contrast agent reservoir, wherein the contrast agent reservoir is in communication with the chamber.

Example 65 may optionally include at least in part the system of example 1, wherein at least one of the following components of the system are MRI compatible so that the system can be used with MR-guided interventional procedures:

the access needle;

the chamber; or

the guide wire.

Example 66 can include, or can optionally be combined with any portion or combination of any portions of any one or more of Examples 1-65 to include, subject matter that can include means for performing any one or more of the functions of Examples 1-65, as well devices, systems, structures, components, compositions, materials, shapes, contours, and sizes. Moreover, any of the means discussed through out examples 1-65 may optionally be performed by any structure, component, device or system discussed throughout this disclosure or any disclosure incorporated by reference herein.

### Example Set No. 67

An aspect of an embodiment of the present invention solves the clinical need to improve the method used to obtain femoral access and precisely guide the operator to puncture the common femoral artery. This device and methodology allows the operator to locate the initial puncture site using fluoroscopically defined bony landmarks (e.g., middle of the femoral head) followed by puncture of the artery with a small, 21 gauge needle while allowing the operator to confirm the location within the common femoral artery by injecting contrast under fluoroscopy. If the operator confirms the proper site in the common femoral artery, a small guide wire can then be advanced through a channel installed in the plunger of the contrast-delivery syringe, into the barrel of the syringe, and then out the distal tip of the needle. Thereafter, the needle and syringe can be removed and replaced by a sheath. However, if the angiogram demonstrates improper position, the needle can be withdrawn and another attempt made leaving behind only a 21 gauge needle puncture and thus minimizing the likelihood of a significant bleed. The entire unit is easy to manipulate and does not add significant time to the procedure, an important feature that will appeal to the temperament of invasive cardiologists.

Design Principles:

1. Start with a 10 ml syringe.
2. A needle is incorporated into the plunger of the syringe. A needle with dimensions suitable for this purpose is Galt catalog number NDL-100-21. The plungers of most disposable plastic syringes have an x-shaped cross section, so the needle is bonded into one of the axial channels, with the proximal end of the hub flush with the top of the plunger. The distal end of the needle pokes through the bottom of the plunger.
3. The distal end of the barrel is modified to have a deeper conical shape, in order to funnel the guide wire out of the port first time, every time. The Luer lock on the end of the syringe is replaced with a Luer slip fitting to allow for rapid attachment/detachment of the needle. In the prototype device, a plastic funnel was used for this purpose,
Qosina catalog number 20046.
4. Finger loop fittings are slid onto the proximal ends of the barrel and the plunger, to make for easy ergonomic use of the syringe during access procedures.

Design Details

We began the prototyping process using a 10 ml plastic syringe. A syringe of a different volume is also easily used. The idea is to have a Luer slip fit on the output port of the syringe rather than a Luer lock, in order to minimize the time needed for attaching needles, etc. to it. Therefore, when the relatively flat distal end of the syringe is cut off, the funnel-shaped replacement must have the same kind of Luer slip port on it. A funnel angle on the order of about 45 to 50 degrees is satisfactory. Steeper or shallower angles will also work. The reason for the angulation is to cause the guide wire to pass easily through the syringe exit port without doubling back on itself inside the bottom of the syringe. Shown here are the syringe barrels with (upper) and without (lower) the tapered funnel attached.

The next step is to install a 21 Gauge Galt (or equivalent) introducer needle in the plunger. The plungers of such syringes typically have a simple cruciform shape and a flat plastic top against which the user presses their thumb during an injection. We forced the tip of the needle all the way through the seal at the bottom of the syringe, such that the upper lip of the needle's female entry port was flush with the top of the plunger. We then cut out one quadrant of the plunger's top so that the needle's entry port would be contained within the cruciform's cross sectional area. We then glued it in place.

Plungers with and without the needle installed are shown here.

The plunger can then be inserted inside the barrel of the syringe. Since the tip of the needle integral to the plunger sticks only 2 or 3 mm past the distal end of the plunger's seal, it does not bottom out on the inside surface of the tapered funnel at the distal end of the syringe. Hence, the guide wire cannot bind inside of that region of the barrel when the plunger is pushed down to near or at the end of its internal travel within the barrel.

Finger loops can then be press fitted onto the proximal ends of the barrel and plunger of the modified syringe. The finger loops are helpful to the operator, since they make it possible to either inject or withdraw the plunger using only one hand, because the fingers can work against either the upper or lower inner surfaces of the finger loops. A 21 gauge Galt needle can be slip-fitted onto the distal end of the tapered funnel and the device will be ready for femoral access.

The devices, systems, compositions, computer program products, and methods of various embodiments of the invention disclosed herein may utilize aspects disclosed in the following references, applications, publications and patents and which are hereby incorporated by reference herein in their entirety:
1. U.S. Pat. No. 4,274,408, Nimrod, B., “Method For Guide-Wire Placement and Novel Syringe Therefore”, Jun. 23, 1981;
2. U.S. Pat. No. 4,813,938, Raulerson, J., “Catheter Introduction Syringe”, Mar. 21, 1989;
3. U.S. Pat. No. 5,045,065, Raulerson, J., “Catheter Introduction Syringe”, Sep. 3, 1991;
4. U.S. Pat. No. 5,879,338, Mahurkar, S., “Needle-Syringe Assembly for Guidewire Insertion”, Mar. 9, 1999;
5. U.S. Pat. No. 5,579,780, Zadini, et al., “Manual Guidewire Placement Device”, Dec. 3, 1996;
6. U.S. Pat. No. 5,873,841, Brannon, J., “Syringe with Decelerating Device”, Feb. 23, 1999;
7. U.S. Pat. No. 6,013,037, Brannon, J., “Syringe with Conduit”, Jan. 11, 2000;
8. U.S. Pat. No. 4,468,224, Enzmann, et al., “System and Method for Catheter Placement in Blood Vessels of a Human Patient”, Aug. 28, 1984;
9. U.S. Pat. No. 5,380,290, Makower, et al., “Body Access Device”, Jan. 10, 1995;
10. U.S. Pat. No. 6,533,759 B1, Watson, et al., “Flash Chamber with a Self Closing Valve for Use with a Catheter”, Mar. 18, 2003;
11. U.S. Patent Application Publication No. US 2010/0076406 A1, Raulerson, J., “Co-Axial Catheter and Method of Implanting Same”, Mar. 25, 2010; U.S. patent application Ser. No. 12/611,524, filed Nov. 3, 2009;
12. U.S. Pat. No. 3,703,174, Smith, G., “Method and Apparatus for Catheter Injection”, Nov. 21, 1972;
13. U.S. Pat. No. 3,792,703, Moorehead, “Catheter Placement Unit”, Feb. 19, 1974;
14. U.S. Pat. No. 3,903,885, Fuchs, H., “Device for Introducing Flexible Catheters”, Sep. 9, 1975;
15. U.S. Pat. No. 7,285,097 B2, Tenerz, et al., “Technique to Confirm Correct Positioning with Respect to Arterial Wall”, Oct. 23, 2007;
16. U.S. Pat. No. 7,201,725 B1, Cragg, et al., “Device and Method for Determining a Depth of an Incision”, Apr. 10, 2007;
17. U.S. Pat. No. 7,104,981 B2, Elkins, et al., “Apparatus and Method for Inserting an Intra-Aorta Catheter Through a Delivery Sheath”, Sep. 12, 2006;
18. U.S. Pat. No. 7,094,209 B2, Egnelov, et al., “Method for Introducer Replacement”, Aug. 22, 2006;
19. U.S. Pat. No. 7,073,509 B2, Tenerz, et al., “Technique to Confirm Correct Positioning of Arterial Wall Sealing Device”, Jul. 11, 2006;
20. U.S. Pat. No. 7,048,711 B2, Rosenman, et al., “Drug Delivery Catheter and Steering Guide Catheter Combination”, May 23, 2006;
21. U.S. Pat. No. 7,008,442 B2, Brightbill, J., “Vascular Sealant Delivery Device and Sheath Introducer and Method”, Mar. 7, 2006;
22. U.S. Pat. No. 6,994,700 B2, Elkins, J., et al., “Apparatus and Method for Inserting an Intra-Aorta Catheter Through a Delivery Sheath”, Feb. 7, 2006;
23. U.S. Pat. No. 6,949,107 B2, McGuckin, Jr., et al., “Injection Method for Locating Vessel Lumen”, Sep. 27, 2005;
24. U.S. Pat. No. 6,884,235 B2, McGuckin, Jr., et al., “Introducer Sheath with Retainer and Radiopaque Insert”, Apr. 26, 2005;
25. U.S. Pat. No. 6,836,687 B2, Kelley, et al., “Method and System for Delivery of a Medical Electrical Lead Within a Venous System”, Dec. 28, 2004;
26. U.S. Pat. No. 6,674,464 B2, McGuckin, Jr., et al., “Introducer Sheath with Retainer”, Jul. 20, 2004;
27. International Patent Application Publication No. WO 98/07372, Hammerslag, J., “Surface Opening Adhesive Sealer”, Feb. 26, 1998; International Application No. PCT/US97/14279, filed Aug. 13, 1997;
28. International Patent Application Publication No. WO 97/11737, Erbel, et al.,
   “System for Actively Supporting the Flow of Body Fluids”, Apr. 3, 1997; International Patent Application No. PCT/DE96/01834, filed Sep. 26, 1996;
29. International Patent Application Publication No. DE 4319033 C1, Schmidt, K., “Seldinger Device with Vein Catheterisation”, Jun. 30, 1994;
30. Eckhardt, et al., “Inadvertent Carotid Artery Cannulation During Pulmonary Artery Catheter Insertion”, Journal of Cardiothoracic and Vascular Anesthesia, Vol. 10, No. 2, February 1996, pp 283-290;
31. Stein, et al., “Inadvertent Intraarterial Placement of a Sheath Introducer while Using the Raulerson Syringe”, Anesthesiology, Vol. 7, No. 3, March 1991, pp 633-634;
32. Frederickson, et al., “A Prospective Randomized Comparison of Ultrasound and Neurostimulation as Needle End Points for Interscalene Catheter Placement”, Anesthesia & Analgesia, Vol. 108, No. 5, May 2009, pp 1695-1700;
33. Fyntanidou, et al., “The Use of Central Venous Catheters During Emergency Prehospital Care: A 2-Year Experience”, European Journal of Emergency Medicine, Vol. 16, No. 4, 2009, pp 194-198;
34. Oliver, et al., “The Incidence of Artery Puncture with Central Venous Cannulation Using a Modified Technique for Detection and Prevention of Arterial Cannulation”, Journal of Cardiothoracic and Vascular Anesthesia, Vol. 11, No. 7, December 1997, pp 851-855;
35. Bauriedel, et al., “Sheath Introducer Technique for Recanalizing Total Occlusions of the Superficial Femoral Artery”, Catherization and Cardiovascular Diagnosis 25:66-70 (1992);
36. U.S. patent application Ser. No. 12/741,710 entitled “Steerable Epicardial Pacing Catheter System Placed via the Subxiphoid Process,” filed May 6, 2010, U.S. Patent Application Publication No. 2010/0241185, Sep. 23, 2010;
37. International Patent Application No. PCT/US2008/082835 entitled “Steerable Epicardial Pacing Catheter System Placed via the Subxiphoid Process,” filed Nov. 7, 2008, International Patent Application Publication No. WO 2009/062061, May 14, 2009;
38. International Patent Application No. PCT/US2010/033189 entitled “Access Trocar and Related Method Thereof,” filed Apr. 30, 2010;
39. U.S. patent application Ser. No. 12/760,837 entitled “Coaxial Catheter Systems for Transference of Medium,” filed Apr. 15, 2010;
40. U.S. patent application Ser. No. 11/191,676 entitled “Coaxial Catheter Systems for Transference of Medium,” filed Jul. 28, 2005; U.S. Pat. No. 7,727,225 Jun. 1, 2010;
41. International Patent Application No. PCT/US2005/026738 entitled “Coaxial Catheter Systems for Transference of Medium,” filed Jul. 28, 2005, International Patent Application Publication No. WO06/15091, Feb. 9, 2006;
42. U.S. patent application Ser. No. 12/625,153 entitled “Blood Flow Bypass Catheters and Methods for the Delivery of Medium to the Vasculature and Body Ducts,” filed Nov. 24, 2009;
43. U.S. patent application Ser. No. 11/884,421 entitled “Blood Flow Bypass Catheters and Methods for the Delivery of Medium to the Vasculature and Body Ducts,” filed Aug. 15, 2007, U.S. Patent Application Publication No. 2008/0262467, Oct. 23, 2008;
44. International Patent Application No. US2006/005876 entitled “Blood Flow Bypass Catheters and Methods for the Delivery of Medium to the Vasculature and Body Ducts,” filed Feb. 16, 2006, International Patent Application Publication No. WO06/089243, Aug. 24, 2006;
45. U.S. patent application Ser. No. 12/532,233 entitled “Electrode Catheter for Ablation Purposes and Related Method Thereof,” filed Sep. 21, 2009, U.S. Patent Application Publication No. 2010/0211064, Aug. 19, 2010;
46. International Patent Application No. PCT/US2008/057626 entitled “Electrode Catheter for Ablation Purposes and Related Method Thereof,” filed Mar. 20, 2008, International Patent Application Publication No. WO 2008/118737, Oct. 2, 2008;
46. U.S. patent application Ser. No. 12/530,938 entitled “Epicardial Ablation Catheter and Method of Use,” filed Sep. 11, 2009, U.S. Patent Application Publication No. 2010/0114093, May 6, 2010;
47. International Patent Application No. PCT/US2008/056816 entitled “Epicardial Ablation Catheter and Method of Use,” filed Mar. 13, 2008, International Patent Application Publication No. WO 2008/112870, Sep. 18, 2008;
48. U.S. patent application Ser. No. 12/530,830 entitled “Access Needle Pressure Sensor Device and Method of Use,” filed Sep. 11, 2009, U.S. Patent Application Publication No. 2010/0094143, Apr. 15, 2010;
49. International Patent Application No. PCT/US2008/056643 entitled “Access Needle Pressure Sensor Device and Method of Use,” filed Mar. 12, 2008, International Patent Application Publication No. WO 2008/115745, Sep. 25, 2008;
50. U.S. patent application Ser. No. 12/304,801 entitled “Closure Device for Skull Plates and Related Method Thereof,” filed May 18, 2009, U.S. Patent Application Publication No. 2010/0042158, Feb. 18, 2010;
51. International Patent Application No. PCT/US2007/014881 entitled “Closure Device for Skull Plates and Related Method Thereof,” filed Jun. 26, 2007, International Patent Application Publication No. WO 2008/02595, Jan. 3, 2008;
52. U.S. patent application Ser. No. 12/513,258 entitled “Means and Methods for Cytometric Therapies,” filed May 1, 2009, U.S. Patent Application Publication No. 2010/0210927, Aug. 19, 2010;
53. International Patent Application No. PCT/US2007/023047 entitled “Means and Methods for Cytometric Therapies,” filed Nov. 1, 2007, International Patent Application No. WO 2008/057370, May 15, 2008;
54. U.S. patent application Ser. No. 12/375,139 entitled “System and Method for Intracranial Implantation of Therapeutic or Diagnostic Agents,” filed Jan. 27, 2009, U.S. Patent Application Publication No. 2009/0192487, Jul. 30, 2009;
55. International Patent Application No. PCT/US2007/016256 entitled “System and Method for Intracranial Implantation of Therapeutic or Diagnostic Agents,” filed Jul. 18, 2007, International Patent Application No. WO 2008/013709, Jan. 31, 2008;
56. U.S. patent application Ser. No. 12/160,378 entitled “Multi-Port Catheter System with Medium Control and Measurement Systems for Therapy and Diagnosis Delivery,” filed Aug. 1, 2008, U.S. Patent Application Publication No. 2009/0048577, Feb. 19, 2009;
57. International Patent Application No. PCT/US2007/000353 entitled “Multi-Port Catheter System with Medium Control and Measurement Systems for Therapy and Diagnosis Delivery,” filed Jan. 9, 2007, International Patent Application Publication No. WO 2007/081842, Jul. 19, 2007;
58. International Patent Application No. US2006/013621 entitled “Catheter Systems for Delivery of Agents and Related Method Thereof,” filed Apr. 12, 2006, International Patent Application Publication No. WO06113267, Oct. 26, 2006;
59. U.S. patent application Ser. No. 11/105,166 entitled “Catheter Systems for Delivery of Agents and Related Method Thereof,” filed Apr. 13, 2005, U.S. Patent Application Publication No. 2005/0245896, Nov. 3, 2005; U.S. Pat. No. 7,670,327, issued Mar. 2, 2010;
60. U.S. patent application Ser. No. 10/985,340 entitled “Catheter Navigation within an MR Imaging Device,” filed Nov. 10, 2004, U.S. Patent Application Publication No. 2005/0119556, Jun. 2, 2005;
61. U.S. patent application Ser. No. 10/429,524 entitled “Catheter Navigation within an MR Imaging Device,” filed May 5, 2003, U.S. Patent Application Publication No. 2003/0195412, Oct. 16, 2003; U.S. Pat. No. 6,834,201, issued Dec. 21, 2004;
62. International Patent Application No. PCT/US2002/002363 entitled “Catheter Navigation within an MR Imaging Device,” filed Jan. 28, 2002;
63. U.S. patent application Ser. No. 09/772,188 entitled “Catheter Navigation within an MR Imaging Device,” filed Jan. 29, 2001;
64. U.S. patent application Ser. No. 10/444,884 entitled “Cell Delivery Catheter and Method,” filed May 23, 2003, U.S. Patent Application Publication No. 2003/0204171, Oct. 30, 2003;
65. U.S. patent application Ser. No. 09/574,857 entitled “Cell Delivery Catheter and Method,” filed May 19, 2000, U.S. Pat. No. 6,599,274, issued Jul. 29, 2003;
69. U.S. application Ser. No. 09/548,110 entitled “Multi-Probe System,” filed Apr. 12, 2000, U.S. Pat. No. 6,626,902, issued Sep. 30, 2003;
70. International Patent Application No. US99/24253 entitled “Mri And Magnetic Stereotaxis Surgical System,” filed Oct. 15, 1999, International Patent Application Publication No. WO00/23000, Apr. 27, 2000;
71. U.S. patent application Ser. No. 09/174,189 entitled “Combined Magnetic Resonance Imaging and Magnetic Stereotaxis Surgical Apparatus and Processes,” filed Oct. 16, 1998, U.S. Pat. No. 6,298,259, issued Oct. 2, 2001;
72. International Patent Application No. US99/17880 entitled “MR-Visible Device for Magnetic Stereotaxis Neurological Interventions,” filed Aug. 6, 1999, International Patent Application No. WO00/07652, Feb. 17, 2000;
73. U.S. patent application Ser. No. 09/131,031 entitled “MR-Visible Medical Device for Neurological Interventions Using Nonlinear Magnetic Stereotaxis and a Method Imaging,” filed Aug. 7, 1998, U.S. Pat. No. 6,272,370, issued Aug. 7, 2001;
74. U.S. patent application Ser. No. 09/114,414 entitled “Magnetic Stereotactic System for Treatment Delivery,” filed Jul. 13, 1998, U.S. Pat. No. 6,216,030, Apr. 10, 2001;
75. U.S. patent application Ser. No. 08/464,279 entitled “Magnetic Stereotactic System for Treatment Delivery,” filed Jun. 5, 1995, U.S. Pat. No. 5,707,335, issued Jan. 13, 1998;
76. U.S. patent application Ser. No. 08/096,214 entitled “Magnetic Stereotactic System for Treatment Delivery,” filed Jul. 19, 1993, U.S. Pat. No. 5,779,694, filed Jul. 14, 1998;
77. U.S. patent application Ser. No. 07/904,032 entitled “Magnetic Stereotactic System For Treatment Delivery,” filed Jun. 25, 1992;
78. U.S. patent application Ser. No. 07/463,340 entitled “Magnetic Stereotactic System for Treatment Delivery,” filed Jan. 10, 1990, U.S. Pat. No. 5,125,888 Jun. 30, 1992.

Unless clearly specified to the contrary, there is no requirement for any particular described or illustrated activity or element, any particular sequence or such activities, any particular size, speed, material, duration, contour, dimension or frequency, or any particularly interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. It should be appreciated that aspects of the present invention may have a variety of sizes, contours, shapes, compositions and materials as desired or required.

In summary, while the present invention has been described with respect to specific embodiments, many modifications, variations, alterations, substitutions, and equivalents will be apparent to those skilled in the art. The present invention is not to be limited in scope by the specific embodiment described herein. Indeed, various modifications of the present invention, in addition to those described herein, will be apparent to those of skill in the art from the foregoing description and accompanying drawings. Accordingly, the invention is to be considered as limited only by the spirit and scope of the following claims, including all modifications and equivalents.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particularly interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

1-65. (canceled)

66. An access system that provides the capability to inject a contrast agent while simultaneously navigating a guide wire into a vicinity of a vasculature, a non-vasculature structure, and/or the vasculature of a subject through a tip of an access needle, said access system comprising:
said access needle;
said guide wire; and
a chamber, wherein said chamber is configured to allow said guide wire to pass through said chamber and said access needle while simultaneously injecting said contrast agent from said chamber into the subject to guide said access needle beneath the skin, in the vicinity of a vasculature, the non-vasculature structure, and/or in the vasculature.

67. The system of claim 66, wherein the system is configured for fluoroscopy to be used with said contrast agent to navigate said guide wire and said access needle beneath the skin, in the vicinity of a vasculature, the non-vasculature structure, and/or in the vasculature.

68. The system of claim 66, wherein said access needle has more than one internal lumen to facilitate the simultaneous or sequential delivery of more than one diagnostic or therapeutic agent, fluid, or device.

69. The system of claim 66, wherein said access system further comprises:
a sheath, wherein said sheath is configured for sliding over said access needle after said access needle punctures the vasculature.

70. The system of claim 66, wherein said chamber comprises a distal end and a first proximal end and a second proximal end;
said access needle is in communication with said chamber distal end;
said chamber first proximal end is configured for receiving said guide wire to allow for said passing of said guide wire through said chamber distal end and into said access needle.

71. The system of claim 70, further comprising a contrast agent reservoir, wherein said contrast agent reservoir is in communication with said chamber.

72. The system of claim 71, wherein said contrast agent reservoir comprises:
a plunger, wherein said plunger comprises a distal end and a proximal end; and
a syringe, wherein said syringe comprises a distal end and a proximal end, whereby said proximal end has an opening configured for receiving said plunger.

73. The system of claim 70, wherein said chamber is a y-tube, said chamber first proximal end and said chamber second proximal end correspond to a first proximal arm and a second proximal arm of said y-tube respectively and said chamber distal end corresponds to a distal arm of said y-tube.

74. The system of claim 73, wherein an angle between said first proximal arms and said second proximal arms is less than about 90 degrees.

75. The system of claim 70, wherein said chamber further comprises a third proximal end configured for at least one of other material or other liquid to be injected, or other device to be passed, into the body in conjunction with the delivery of said contrast agent and passage of said guide wire.

76. The system of claim 75, wherein, at least one of:
said other material further comprises at least one of powders of diagnostic or therapeutic agents or slurries of diagnostic or therapeutic agents;
said other liquids further comprises at least one of antibiotics, anticoagulants, or thrombolytic agents; and
said other device further comprises at least one of thrombectomy devices, closure devices, dilators, or sheaths.

77. The system of claim 70, further comprising a seal disposed in said chamber first proximal end and an introducer conduit in communication with said seal, whereby said introducer conduit configured to allow passing of said guide wire through said seal and into said chamber.

78. The system of claim 70, further comprising a valve disposed on said chamber first proximal end, wherein said valve can be closed to seal off said chamber first proximal end while said guide wire or other tool is being passed through said valve.

79. The system of claim 70, wherein said chamber proximal end is configured to receive a plunger; wherein said plunger comprises a distal end and a proximal end.

80. The system of claim 79, wherein said plunger comprises a introducer conduit extending from said plunger proximal end to said plunger distal end, wherein said introducer conduit comprises a proximal end configured to receive said guide wire and a distal end configured to pass said guide wire into said chamber.

81. The system of claim 80, wherein at least one of the following components of said system are transparent and visible from outside of the system:
said access needle;
said chamber;
said introducer conduit;
said plunger; or
said guide wire.

82. The system of claim 80, wherein at least one of the following components of said system are MRI compatible so that said system can be used with MR-guided interventional procedures:
said access needle;
said chamber;
said introducer conduit;
said plunger; or
said guide wire.

83. A method for using an access system that provides the capability to inject a contrast agent while simultaneously navigating a guide wire into a vasculature, a non-vasculature structure, and/or a vicinity of a vasculature of a subject through a tip of an access needle, said method comprising:
advancing said access needle of said system through the skin of a subject;
determining if said access needle is in the correct location; and:
if said access needle is in the correct location then further advancing said access needle of said system until said access needle has accessed the vasculature, the non-vasculature structure, and/or the vicinity of the vasculature of the subject; or
if said access needle is not in the correct location withdrawing or repositioning said access needle and then determining if said access needle in the correct location.

84. The method of claim 83, wherein said determining if said access needle is in the correct location further comprises passing said guide wire through a chamber and said access needle while simultaneously injecting said contrast agent from said chamber into the subject to guide said access needle beneath the skin, in the vicinity of the vasculature, the non-vasculature structure, and/or in the vasculature.

85. A method for using an access system that provides the capability to inject contrast agent while simultaneously navigating a guide wire into a vicinity of a vasculature, a non-vasculature structure, and/or the vasculature of a subject through a tip of an access needle, said access method comprising:
passing said guide wire through a chamber and said access needle while simultaneously injecting contrast agent from said chamber into the subject to guide said access needle that is inserted beneath the skin, in the vicinity of a vasculature, non-vasculature and/or in the vasculature.
